(19) United States
(12) Patent Application Publication
(10) Pub. No.: US 2018/0036374 A1
(43) Pub. Date: Feb. 8, 2018

(54) Single Walled Carbon Nanotube Polynucleotide Complexes and Methods Related Thereto

(71) Applicant: Ensysce Biosciences, Inc., San Diego, CA (US)

(72) Inventor: Lynn D. Kirkpatrick, Houston, TX (US)

(21) Appl. No.: 15/787,926

(22) Filed: Oct. 19, 2017

Related U.S. Application Data
(62) Division of application No. 15/041,989, filed on Feb. 11, 2016, now abandoned.
(60) Provisional application No. 62/114,853, filed on Feb. 11, 2015.

(51) Int. Cl. A61K 38/17; A61K 9/00; A61K 48/00

(52) U.S. Cl. CPC A61K 38/1774; A61K 35/17; A61K 9/0019; A61K 9/0092; A61K 48/00

(57) ABSTRACT

The present invention provides single-walled carbon nanotube formulations for the delivery of bioactive agents including large polynucleotides encoding therapeutic proteins into hard-to-transfect cells and methods of making such single-walled carbon nanotube formulations.

### CROSS REFERENCE TO RELATED APPLICATIONS

This application is a divisional of, and claims priority to, U.S. application Ser. No. 15/041,989, filed Feb. 11, 2016, published as US 2016/0228544 on Aug. 11, 2016, which claims priority to U.S. Provisional Application Ser. No. 62/114,853 filed Feb. 11, 2015, all of which are incorporated herein by reference.

### FIELD OF THE INVENTION

This invention relates generally to compositions and methods for delivering oligonucleotides encoding therapeutic and immunogenic proteins using carbon nanotubes into hard-to-transfect primary cells including mononuclear cells.

### BACKGROUND OF THE INVENTION

Human PBMCs offer subpopulations of cells involved in specialized effector and regulatory functions in response to immunological stimuli including T lymphocytes responsible for the cellular immune response. T cell subsets can feature lifetime immune memory following presentation of an antigen and because of their easy accessibility and immunological specificity are attractive targets for gene therapy. Thus, making cancer specific T cells is a potential way of providing patients with lifelong protection against their cancer. Treatment of B cell leukemia using T cells that express Chimeric Antigen Receptors (“CAR”) has produced remarkable examples of cures of an otherwise untreatable disease, and has the potential to have a major impact on therapy of other cancers. See Kalos et al. Sci Transl Med. Vol. 3(95) (2011) pp. 1-21. However, primary cells have traditionally presented transfection difficulties and gene transfer to human PBMCs for purposes of therapy has proven surprisingly difficult. The CAR approach is currently limited by the difficulty of efficient gene transfer into primary T cells. Certain viral vectors, including retroviral and adenoviral vectors, have been used for gene transfection into lymphoid cells because of their ability to transduce hematopoietic cells. However, viral gene transfer into lymphoid cells has major disadvantages due to limitations on the length of DNA that can be inserted, difficulties in consistent production of vectors, immunogenicity of viral proteins, required containment and the need for highly skilled technicians. Furthermore, application of retroviral vectors in the clinical setting is hindered by oncogenicity concerns and a poor ability to target specific cell populations.

Nonviral gene delivery systems include the use of DEAE-dextran or calcium phosphate co-precipitation, cationic lipid “liposome” delivery, direct microinjection into cultured cells, electroporation and biolistic particles. While these procedures generally allow flexibility with respect to the nucleic acid sequences that can be inserted into cells, inherent disadvantages include low efficiency of transfection, significant loss of cell viability, laborious procedures and the need of specialized instrumentation. Because of this, attempts to facilitate transfer of DNA into hard-to-transfect primary T or B lymphocytes using nonviral vectors have met with limited success. Several rate-limiting steps, such as suboptimal attachment and internalization through the cell membrane, and/or inadequate release of vector-DNA complexes from the endosomes and transport of DNA to the nucleus, may be partially responsible for the inefficiency of nonviral vectors for gene delivery to lymphoid cells. Approaches to modify nonviral vectors by covalent attachment of various ligands to facilitate entry of DNA into cells via specific receptors have provided a more efficient gene delivery to T lymphoblastoid cell lines, however, the efficiency of gene expression in primary unstimulated PBMC is low.

Therefore the development of simple, efficient nonviral gene transfer technologies for T lymphocytes would facilitate T lymphocyte-based clinical applications. A number of studies by the present inventors and others have demonstrated the ability of functionalized single wall carbon nanotubes (“f-SWCNT”) to transfect immortalized cells. Karmakar et al. used ethylenediamine functionalized SWCNT to successfully transfect MCF7 breast cancer cells with plasmid coding for a p53-GFP fusion protein. See Karmakar et al. International Journal of Nanomedicine vol. 6 (2011) 1045-1055. Liu et al have reported f-SWCNT transfection of siRNA into PBMCs. See Liu et al. Angewandte Chemie Int. Ed. 46 (2007) 2023-2027. Others have demonstrated that f-SWCNT can be safely used for therapeutic drug delivery, imaging and tumor ablation.

However, what are needed are simple, safe and effective methods and formulations that enable transfection of relatively large therapeutic protein encoding oligonucleotides into primary PBMC both in vitro and in vivo using un-functionalized SWCNT as a delivery vehicle such that the elusive ideal of conveniently, reliably and safely generating transformed PBMC populations can be achieved and the therapeutic potential realized.

### BRIEF SUMMARY OF THE INVENTION

The present invention is directed to a method for transfection of hard-to-transfect cells with oligonucleotides using pristine single wall carbon nanotubes (“SWCNT”) as a delivery vehicle. Provided herein are formulations and procedures that enable pristine non-functionalized SWCNT to complex with oligonucleotides encoding therapeutic proteins without shearing of the oligonucleotides and further provides SWCNT-nucleic acid complexes able to effectively transfect hard-to-transfect cells including PBMC in vitro and in vivo.

### BRIEF DESCRIPTION OF THE DRAWINGS

For a fuller understanding of the nature and advantages of the present invention, reference should be had to the following detailed description taken in connection with the accompanying drawings, in which:

FIG. 1 depicts Amplitude profile by Atomic Force Microscopy image of a SWCNT/TCRα RNA/PEG complex.

FIG. 2 depicts a height profile by Atomic Force Microscopy image of a SWCNT/TCRα RNA/PEG complex.

FIG. 3 depicts a length analysis by Atomic Force Microscopy image of a SWCNT/TCRα RNA/PEG complex.

FIG. 4 shows the results of agarose gel electrophoresis used to determine if the RNA was sheared through processing or if it remained intact. Samples defined as T1 11/3/14 and 12/8/14 were prepared using previously reported siRNA sonication procedures using 100% power and 15 second pulses for a total of 4 min. Altering the sonication procedure to use 10% power but longer sonication time for overall higher energy input provided a SWCNT/RNA complex with superior RNA loading and the RNA remained intact (T1 12/22/14)

FIG. 5 shows the results transfection of primary PBMC with the SWCNT/RNA/PEG complex T1 12/22/14 (prepared with 50 μM PEG compared to a second SWCNT/RNA/PEG complex prepared with 1600 μM PEG, T2. Cells were imaged using Cell Titre Blue to measure cell growth.

FIG. 6A provides confocal images of Human PBMC exposed to Cy-3 siEGFR for 24 hr. FIG. 6B provides confocal images of human PBMC exposed to SWCNT complexed siEGFR formulated for in vitro transfection for 24 hr. The insert illustrates single cells at 10× magnification.

FIG. 7A provides confocal images of human PBMC exposed to SWCNT/Cy-3 siEGFR formulated for in vitro delivery (5 μg/mL SWCNT) showing intracellular delivery. FIG. 7B provides confocal images of human PBMC exposed to Cy-3 siEGFR in a liposomal formulation for 2, 6 and 24 hr. Color images showed that without the SWCNT, agglomerated liposomes were located extracellularly.

FIG. 8 shows quantitation of SWCNT in PBMC after in vivo intravenous injection.

### DETAILED DESCRIPTION

Certain of the present inventors have shown that SWCNT are excellent vehicles for delivering siRNA into mammalian cells in culture and tumors and importantly, have also determined the SWCNT rapidly and effectively transfect circulating PBMC where lipid delivery platforms have struggled. Numerous studies have shown that SWCNT are well tolerated and can be used safely for in vivo delivery. The advantages of using SWCNT as a delivery vehicle include their large surface area relative to volume for payload attachment, inert non-toxic carbon backbone structure, ability to protect the payload while circulating, effective tissue delivery with elimination through the bile and urine, and the pharmaceutical stability of the SWCNT/payload complex. Provided herein are novel formulations of SWCNT/gene complexes for transfection of PBMC with genetic material both in vitro and in vivo, which previously had not been possible.

The SWCNT delivery platform for transfection of genes or gene products into human PBMC, including T cells, could eliminate viral based transfection vehicles and their potential for adverse effects. Additionally using SWCNT for the delivery of the CAR technology could provide in vivo transfection rather than having to manufacture the T-cells ex vivo, thus obviating the need for expensive centralized GLP cell transfection facilities and increasing the availability of this exciting new to therapy to a wider population.

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must be noted that, as used herein, and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The term “agglomeration,” as used herein, refers to the formation of a cohesive mass of subunits such as carbon nanotubes held together by relatively weak forces such as, for example, van der Waals forces or capillary action, that can be broken during processing resulting in a group of individual subunits. The mass of subunits resulting from agglomeration is an “agglomerate.”

As used herein, the term “aggregation” refers to the formation of a discrete group of subunits such as carbon nanotubes in which the forces holding the individual subunits together are not easily broken. For example, carbon nanotubes bundles can be strongly bonded together by, for example, covalent bonds. The discrete group of subunits is called an “aggregate.”

As used herein, the term “bioactive agent” refers to a compound utilized to image, impact, treat, combat, ameliorate, prevent, and/or improve, an unwanted condition or disease of a patient. The bioactive agent may modulate any number of biological functions in the cell, such as cell division, cellular infection, cellular expression of cell surface proteins, cellular response to a hormone, among others. The term “bioactive agent” may further refer to polynucleotides including siRNA, small molecules, and polypeptides that cause a metabolic change in a cell, generally by increasing transcription, expression or translocation of one or more genes, or by binding to an expressed protein. Embodiments described herein are not limited to any particular bioactive agent. For example, in various embodiments, the bioactive agent may be a drugs, vaccines, immunological agents, chemotherapeutic agent, diagnostic agent, prophylactic agent, nutraceutical agent, small molecule, nucleic acid, protein, peptide, lipid, carbohydrate, hormone, and combinations thereof.

In particular embodiments, the bioactive agent may be a nucleic acid encoding a full or partial gene product in the form of mRNA, plasmid DNA or cDNA. In certain embodiments, the gene products may allow the expression of cellular components such as receptors, antigens or proteins that regulate cellular activity or growth. Non-limiting working examples provided herein include RNA encoding TCR (vb13.1) alpha and beta with polyA tails of ˜1500 base pairs, cDNA encoding GFP Gene cDNA Clone (Codon Optimized, full-length ORF Clone), expression ready, untagged (Sino Biological Inc.; of about 5657 base pairs), and mRNA encoding EGFP (5meC, Ψ), Enhanced Green Fluorescent Protein mRNA (5-methylcytidine, pseudouridine) TriLink Biotechnologies; of about 996 base pairs). As used herein, the term “large polynucleotide” means polynucleotides of about 300 base pairs (“bp”) or greater that encode a functional protein product. By example, the full length mature mRNA for preproinsulin is 110 aa (including 24 aa signal sequence, the 21 aa A-chain, 35 aa C-peptide, and 30 aa B-chain) would be encoded by a 330 bp mRNA. Large protein encoding polynucleotides are considerably harder to deliver effectively due to problems with shear that are not encountered with small polynucleotides.

The phrase “CAR T-cell therapy” is used herein. A CAR is a protein composed of both a binding domain comprised of an antibody to CD19 antigen found on B cell leukemia and a fusion partner that contains the message that activates the T cell to make it proliferate. Unlike antibody therapies, CAR-modified T cells have the potential to replicate in vivo and long-term persistence could lead to sustained tumor control and obviate the need for repeated infusion of antibody. A non-pathogenic form of HIV virus is currently used to and the vector to transfect the CAR into T cells and there is a concern, since it is there permanently that it could lead to risk of disease development in the cells that are genetically modified. Another disadvantage of using the HIV viral vector is the manufacturing process that is required to prepare the CAR T cells. The T cells have to be prepared ex vivo due to the potential risk of HIV infection, therefore, if it were possible to perform the T cell transfection with a delivery system that did not pose a disease threat in the patient, not only would the therapy become more universal, it would decrease costs and time to provide the treatments.

The term “carbon nanotube” refers to an allotrope of carbon having a cylindrical or tube shape and a diameter of as small as about 1 nm. The term “carbon nanotube” may further include structures that can include, for example, metals, small-gap semiconductors, or large-gap semiconductors such as boron carbon nitride (BCN) nanotubes. The term carbon nanotube as used herein refers to both single-walled carbon nanotubes (SWCNT) and multi-walled carbon nanotubes (MWCNTs). A “single-walled carbon nanotube” or “SWCNT” refers to a carbon nanotube that consists of a one atom thick graphene sheet that has been rolled into a tube. A “multi-walled carbon nanotube” or “MWCNT” refers to a nanotube that include 2 or more one graphene sheets rolled into concentric tubes. The term “carbon nanotubes” may also be graphene in other forms including, for example, graphene spheres or “carbon nanosphere,” which are commonly referred to as buckyballs or fullerenes.

The term “diseased tissue”, as used herein, refers to tissue or cells exhibiting a phenotype that is inconsistent with healthy tissue. For example, “diseased tissue” can include tissues and cells affected by AIDS; pathogen-borne diseases, which can be bacterial, viral, parasitic, or fungal, examples of pathogen-borne diseases include HIV, tuberculosis and malaria; hormone-related diseases, such as obesity; vascular system diseases; central nervous system diseases, such as multiple sclerosis; and undesirable matter, such as adverse angiogenesis, restenosis amyloidosis, toxins, reaction-by-products associated with organ transplants, and other abnormal cell or tissue growth. In some embodiments, “diseased tissue” can refer to tissues and cells associated with solid tumors or other cancerous growth including, but not limited to, those associated with bone, lung, vascular, neuronal, colon, ovarian, breast, and prostate cancer. The term diseased tissue may also refer to tissue or cells of the immune system, such as tissue or cells

An “effective amount” or “therapeutically effective amount” of a composition, as used herein, refers to an amount of a biologically active molecule or complex or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like.

“Gene silencing” as used herein can refer to the suppression of gene expression from, for example, an endogenous gene, exogenous gene, or a transgene, and heterologous gene. Gene silencing may be mediated through processes that affect transcription, through post-transcriptional processing of RNA transcripts, and/or translation of the RNA transcript. In some embodiments, gene silencing can occur through siRNA mediated degradation of mRNA via RNA interference.

The term “knock-down” refers to gene silencing in which the expression of a target gene is reduced as compared with normal gene expression, but gene expression not completely eliminated. Knocking down gene expression can lead to the inhibition of production of the target gene product.

The term “non-functionalized,” as used herein, refers to a chemical composition, such as a carbon nanotube, that is substantially unmodified. As such, each carbon of the carbon nanotube is covalently bonded to a neighboring carbon atom or an unreactive atom such as, for example, hydrogen. Non-functionalized carbon nanotubes do not include reactive functional groups, i.e., a group of atoms capable of forming a covalent bond to a carbon atom or another functional group, covalently bonded to the carbons of the carbon nanotube.

The term “nucleic acid” refers to chemical compositions of monomers having a sugar moiety, a phosphate, and a purine or pyrimidine base and includes deoxyribonucleic acids and ribonucleic acids as well as any single-stranded or double-stranded polymers thereof. Unless specifically limited, the term “nucleic acid” further encompasses known analogs of natural nucleotides that may have similar binding properties with reference to the naturally occurring nucleic acid analog and may be metabolized in a manner similar to naturally occurring nucleotides. Polymeric nucleic acids or “oligonucleotides” are generally referred to as “DNA” when the individual monomers making up the polymeric nucleic acid are deoxyribonucleic acids and “RNA” when the individual monomers making up the polymeric nucleic acid are ribonucleic acids. However, polymeric nucleic acids can include hybrid molecules that can include both deoxyribonucleic acid and ribonucleic acid monomers. Such polymeric nucleic acids may be arranged in any manner. For example, a polymeric nucleic acid may include complementary sequences that allow intermolecular interactions such that the polymeric nucleic acid to include secondary structural elements, or two single stranded polymeric nucleic acid molecules may include complementary sequences that allow intramolecular interactions such that the individual polymeric nucleic acids may bind to one another creating a double stranded polymeric nucleic acid molecule.

The arrangement of nucleic acid monomers in a particular polymeric nucleic acid molecule is commonly referred to as the “sequence” of that nucleic acid molecule. In a phenomenon referred to as “base pairing” a purine nucleic acid monomers, adenine (A) and guanine (G) form hydrogen bonds selectively with pyrimidine nucleic acid monomers thymine (T) and cytosine (C), respectively, to create A-T and G-C “base pairs.” Ribonucleic acids are capable of forming similar base pairs, however, in RNA thymine (T) is replaced with uracil (U) to create an A-U base pair. For DNA and messenger RNA (mRNA), RNA molecules produced as the result of transcription that have a sequence that is complementary to the DNA molecule from which the mRNA is produced, the nucleic acid monomers of a sequence may be arranged in three base pair “codons,” where each codon of the mRNA corresponds to a specific amino acid that transported from the cytosol to a ribosome via transfer RNA (tRNA) during translation.

By “complementary sequence” is meant that the polymeric nucleic acid molecule includes a sequence of individual monomers that allow hydrogen bonds to form between nucleic acid monomers. A “complementary sequence” encompasses a pair of nucleic acid molecules in which each base pair is exactly complementary to the corresponding base pair to the opposing nucleic acid. “Complementary sequence” also encompasses a pair of nucleic acid molecules in which one of the pair include conservatively modified variants of naturally occurring nucleotides and degenerate codon substitutions. For example, degenerate codon substitutions may be achieved by generating sequences in which the third position of one or more selected (or all) codons is substituted with mixed-base and/or deoxyinosine residues.

The term “subject” or “patient,” as used herein, includes human and non-human vertebrates such as wild, domestic, and farm animals.

As used herein, a “pharmaceutically acceptable carrier” is a pharmaceutically acceptable solvent, suspending agent or vehicle for delivering the complexes of the present invention to the patient. The carrier may be liquid or solid and is selected with the planned manner of administration in mind. Examples of pharmaceutically acceptable carriers that may be utilized in accordance with the present invention include, but are not limited to, water, isotonic salt solution, isotonic sugar solution, polyethylene glycol (PEG), aqueous PEG solutions, liposomes, ethanol, organic solvent (e.g. DMSO) dissolved in isotonic aqueous solution, aqueous buffers, oils, and combinations thereof.

The terms “small interfering RNA,” “short interfering RNA,” or “siRNA” refers to short double stranded RNA molecules in which one strand of the double stranded RNA is complementary to a portion of a target gene. An “RNA duplex” or “double-stranded RNA” refers to the structure formed by the complementary pairing between two regions of a RNA molecule. siRNA molecules are typically less than about 30 nucleotides. The siRNA described herein includes double-stranded RNA molecules that are prepared from unmodified, naturally occurring RNA bases as well as siRNA that, for example, include non-naturally occurring RNA base pairs or are chemically-modified siRNA or otherwise stabilized siRNA. siRNA can also include siRNA that is specifically designed to target a specific gene (“targeting siRNA”) and siRNA having a randomly generated sequence (“non-targeting siRNA”). RNA interference (“RNAi”) is the process of sequence-specific, posttranscriptional gene silencing initiated by siRNA. RNAi is seen in a number of organisms such as Drosophila, nematodes, fungi and plants and is believed to be involved in anti-viral defense, modulation of transposon activity, and regulation of gene expression. During RNAi, siRNA induces degradation of target mRNA and consequently inhibition of gene expression.

As used herein, the phrase “therapeutic protein encoding oligonucleotide” refers to a DNA or RNA molecule that encodes a protein or polypeptide of sufficient size to provide a therapeutic benefit including as an antigen. As used herein a therapeutic protein encoded by such oligonucleotide will be greater than 30 residues and will thus be encoded by an oligonucleotide of at least 90 base pairs in the case of DNA and 90 bases in the case of mRNA. Based on an average amino acid (“aa”) molecular weight of 110 Da, a 30 residue aa will have a molecular weight of about 3,300 Da. The DNA molecule will encode a start and stop codon and may further include 5′ and 3′ untranscribed or untranslated regions that optimize or enable transcription and post-transcriptional processing of mRNA produced from the DNA as well as translocation to the appropriate cytoplasmic site for translation into protein by either free or membrane bound ribosomes. The mean (full length) protein length for eukaryotic species has been estimated to be around 375 aa (Brocchieri L, Karlin S. Protein length in eukaryotic and prokaryotic proteomes. Nucleic Acids Res. 2005 Jun. 10 33(10):3390-400) with a considerable range.

As used herein a “stable” SWCNT/polynucleotide complex means an aqueous solution of SWCNT/polynucleotide complexes that remains optically clear and does not show aggregation by unaided visual observation, and maintains a SWCNT concentration in solution of 90% or greater as compared with freshly prepared complexes.

Various embodiments described herein are directed to carbon nanotubes (C″NT″), and in some embodiments, the CNT species of single-walled carbon nanotubes (SWCNT), that are useful for delivery of a bioactive agent or a nucleic acid encoding a bioactive agent. In such embodiments, the bioactive agent or a nucleic acid encoding a bioactive agent may coat the CNT or SWCNT by forming non-covalent interactions with the CNT or SWCNT. In certain embodiments, bioactive agent coated CNT or SWCNT may be combined in a pharmaceutical composition that can be administered to a subject to facilitate delivery of the bioactive agent to the subject. Accordingly, some embodiments described herein include pharmaceutical compositions at least including bioactive agent coated carbon nanotube or SWCNT and a pharmaceutically acceptable carrier, and other embodiments include methods for using such pharmaceutical compositions for treating a subject. While such embodiments are not limited to a particular treating a particular disease, in certain embodiments, the disease may be cancer or another disease characterized by abnormal cell growth.

The CNT or SWCNT of various embodiments may be any carbon nanotube or single-walled carbon nanotubes known in the art. In some embodiments, the CNT or SWCNT may have a diameter for from about 0.5 nm to about 1.5 nm, and in other embodiments, the diameter may be about 1 nm. In still other embodiments, the length of the CNT or SWCNT may be about 300 nm or less. For example, in some embodiments, the CNT or SWCNT may have a length of from about 100 nm to about 400 nm, and in other embodiments, the CNT or SWCNT may have a length of about 150 nm to about 300 nm or about 175 nm to about 250 nm. Near-infrared spectral analysis provides a means for determining nanotube size distribution, purity, concentration in solution, and individualization, and in certain embodiments, the CNT or SWCNT may have a strong near-IR spectral transition in, for example, a range of from about 850 nm to about 1600 nm.

The carbon nanotube or SWCNT of embodiments may be derived from any source. For example, in some embodiments, the carbon nanotube or SWCNT may be produced by known methods including, but not limited to, arc discharge, laser evaporation, chemical vapor deposition, and the like, and in other embodiments, as high quality inexpensive carbon nanotube or SWCNT can be prepared using known catalyst chemical vapor deposition methods. In certain embodiments, carbon nanotube or SWCNT may be prepared using the high pressure carbon-monoxide method (HiPco), in which high pressure carbon monoxide (CO) is disproportionated on iron (Fe) nanoparticles formed in the gas phase from iron pentacarbonyl (Fe(CO)₅) decomposition. Without wishing to be bound by theory, the HiPco method may produce relatively small diameter nanotubes.

As used herein, the term “pristine” in the context of a CNT or SWCNT means the CNT is not “functionalized.” As used herein, the term “functionalized” means modified by covalent bonding to a further chemical moiety or non-covalent treatment of the CNT with a chemical moiety that affixes to and remains affixed to the CNT prior to any addition of a polynucleotide. One example of a functionalized CNT by non-covalent means would be pre-treatment of the CNT with a polyethylene glycol analogue or detergent material such as a sodium dodecyl sulfate analogue that remains coated on the CNT. Such coated CNTs are pre-treated in order to solubilize the CNT and prevent aggregation in solution. Because aggregation is prevented, it is clear that the solubilizing compound remains coated on the surface of the CNT. Thus, a pristine CNT is distinctive from functionalized CNT (“f-CNT”) such as, for example, those f-CNTs having been derivatized with covalently bound moieties or pretreated with chemical moieties to which further molecules are or can be chemically added. For example PEG can be covalently added to SWCNT by treatment of the SWCNT with HNO₃—H₂SO₄ followed by oxidization and activation with 1-[3-(dimethylamino) propyl]-3-ethylcarbodiimide hydrochloride [EDC]/N-hydroxysuccinimide [NETS] prior to addition of amino-PEG. Such a CNT is a functionalized-CNT and not a pristine CNT as the terms “functionalized” and “pristine” are used herein.

As further used herein, the term “pristine” in the context of a polynucleotide means the polynucleotide is not “functionalized.” As above, the term “functionalized” means modified by covalent bonding to a further chemical moiety. As used herein, the term “pristine” polynucleotide means the polynucleotide is not grafted to any moiety by which the polynucleotide can be covalently bound to any other molecule. Thus, a pristine polynucleotide is distinctive from functionalized polynucleotides such as, for example, those polynucleotides having been derivatized with covalently bound moieties to which further molecules are chemically added. For example polynucleotides can be covalently modified by addition of end groups such as thiol groups. f-SWCNT such as those that have been coated with polylysine PEG2000-NH2) can then be attached to the thiol modified nucleotides through disulfide bonds by using a sulfo-LC-SPDP (sulfosuccinimidyl 6-(3′-(2-pyridyldithio)propionamido)hexanoate) linker in the presence of EDC and sulfo-NHS. See e.g. Liu, Z. et al. Angew. Chem. Int. Ed. 2007, 46, 2023-2027. Such a polynucleotide is a functionalized polynucleotide and not a pristine polynucleotide as the terms “functionalized” and “pristine” are used herein. In the Liu disclosure, the functionalized polynucleotide is attached to an f-CNT not a pristine CNT as defined herein.

In some embodiments, the pristine SWCNT may be combined with a bioactive agent to make a SWCNT/bioactive agent complex, in which the bioactive agent forms a non-covalent association with the SWCNT, and in other embodiments, the SWCNT may be combined with a bioactive agent to make a SWCNT/bioactive agent complex. In still other embodiments, the SWCNT may be combined with siRNA to make a SWCNT/siRNA complex, in which each delivery vehicle only includes a SWCNT and one or more siRNA molecule associated with the SWCNT. As used herein, the term “SWCNT complexes” shall encompass SWCNT/bioactive agent complexes. The amount of bioactive agent combined with the SWCNT may vary among embodiments and may be determined based on the amount of surface of each SWCNT to be covered by the bioactive agent. For example, in some embodiments, the ratio of complexed to non-complexed surface area on the SWCNT may be selected to provide sufficient coverage to allow the SWCNT to be soluble in solution and provide a therapeutically effective amount of bioactive agent to be delivered. In certain embodiments, less than about 95% of the total surface area of the SWCNT may be in complex with the bioactive active agent and/or surfactant, and in other embodiments, less than about 50% of the surface area of the SWCNT may be in complex with the bioactive agent and/or surfactant. The amounts of SWCNT and bioactive agent or siRNA combined to form the SWCNT complexes may, therefore, vary accordingly. For example in some embodiments, a composition of SWCNT complexes may include about 1 ng/μl to about 10 ng/μl based on the total volume of the composition and about 10 ng/μl to about 40 ng/μl of oligonucleotide. In other embodiments, the SWCNT may be provided in a concentration of about 2 ng/μl to about 5 ng/μl and about 15 ng/μl to about 30 ng/μl of oligonucleotide or about 3 ng/μl of SWCNT and about 25 ng/μl of oligonucleotide.

In other embodiments, the SWCNT/biological agent complex may be subsequently treated with one or more surfactants, and in some embodiments, the surfactant may be a mild detergent that can associate with the SWCNT/biological agent complex and allow improved blood circulation times for the SWCNT/biological agent complex. For example, in particular embodiments, the surfactant may be a polyalkylene oxide such as, for example, PLURONIC™ PEG 5000, PEGS 000 PE (1,2-dimyri stoyl-sn-glycero-3-phosphoethanolamine-N-[methoxy(polyethylene glycol)-5000] (ammonium salt), C18-PMH-mPEG (poly(maleic anhydride-alt-1-octadecene)-poly(ethylene glycol) methyl ether), (or a combination thereof). As used herein the term, “lipid-PEG” or “L-PEG” means a polyethylene glycol having a lipid moiety covalently attached. The concentration of the surfactant may vary in embodiments and may be sufficient to increase blood circulation times without affecting the stability of the SWCNT complexes or the physiological acceptability of the compositions. The addition of a surfactant to 1% to about 15% or about 2% to about 9% of the total solution volume may further increase the blood circulation time of the SWCNT complexes up to about 2-5 fold, up to about 10 fold, or up to 100 fold of a SWCNT complex alone without surfactant. In particular embodiments, the surfactant may be provided at about 3% of the solution.

In some embodiments, the SWCNT/bioactive agent complex or SWCNT/oligonucleotide complex may be prepared in an aqueous buffer that is physiologically acceptable for in vivo or in vitro use, and in other embodiments, the SWCNT complexes may be prepared in a buffer suitable for administration to a mammal such as, for example, a mouse, rabbit, ape, or human. As such, in certain embodiments, the SWCNT complexes may be combined with one or more pharmaceutically acceptable carriers or excipients to produce a pharmaceutical formulation. The carrier or excipient may vary among embodiments and may be selected based on factors including, but not limited to, route of administration, location of the disease tissue, the bioactive substance being delivered, and/or time course of delivery of the bioactive substance. For example, in some embodiments, the pharmaceutically acceptable carrier may be water, and in other embodiments, the pharmaceutically acceptable carrier may be water combined with a physiologic salt to create an aqueous solution that is isotonic to blood serum. In still other embodiments, the pharmaceutical compositions of embodiments can include one or more preservative.

The pharmaceutical compositions of various embodiments may be prepared to deliver of a therapeutically effective amount of the bioactive agent to the subject. For example, in embodiments in which the bioactive agent is a siRNA, an effective amount of the SWCNT/oligonucleotide complex may be an amount sufficient to increase expression of the target protein or receptor in affected tissue. The therapeutically effective amount may vary depending on the type disease being treated, the extent of disease, disease progression, age of the patient, weight of the patient, and the like. In some embodiments, a therapeutically effective amount may be up to about 5 μg/kg or greater. In other embodiments, a therapeutically effective amount may be from about 0.1 μg/kg to about 4 μg/kg or greater, and in still other embodiments, a therapeutically effective amount may be from about 0.5 μg/kg to about 3 μg/kg or about 2.5 μg/kg.

Without wishing to be bound by theory, the SWCNT complexes of various embodiments may be very well tolerated when administered to a patient, such that large doses of a SWCNT complex may be provided to a patient with limited or no adverse side effects. For example, in some embodiments, a dose of greater than 10 mg or greater than 15 mg may be administered to a human without adverse side effects. Accordingly, various embodiments include pharmaceutical compositions prepared for high dose administration of SWCNT complexes.

Further embodiments are directed to methods for delivering a bioactive agent to diseased tissue including the steps of administering a therapeutically effective amount of a SWCNT complex to a patient, methods for treating a disease by administering a pharmaceutical composition including a therapeutically effective amount of a SWCNT complex to a patient in need of treatment, and methods for silencing a targeted gene in vivo including administering a therapeutically effective amount of a SWCNT complex to a patient. Any SWCNT complex including any bioactive agent described herein may be administered as part of a pharmaceutical composition in such methods. In certain embodiments, the SWCNT complex to be delivered may be a SWCNT/oligonucleotide complex. In some embodiments, delivering may include contacting diseased tissue with the bioactive agent, and in some embodiments, delivering may include internalization of the active agent and, in certain embodiments, SWCNT into cells of the diseased tissue. For example, about 0.01% to about 30% of the total SWCNT/oligonucleotide complex can be internalized in vitro in media containing 10% serum after about 1 hour, from about 20% to about 90% of the total SWCNT/oligonucleotide complex can internalized after about 6 hours, and after about 24 hours about 95% of more of the total SWCNT/oligonucleotide complex can be internalized. The bioactive agent may remain in complex with the SWCNT after being internalized by the cell, or in some embodiments, the bioactive agent may dissociate from the SWCNT when internalized by the cell including primary blood mononuclear cells.

A broad range of diseases can be treated using the methods described herein, as SWCNT complexes of various embodiments can function as a serum-insensitive transfection agent to effectuate delivery of various bioactive agents into a cell. For example, in some embodiments, SWCNT/oligonucleotide complex may be used to deliver the oligonucleotide, gene or other gene product to diseased tissues and cells to induce a response, which can effectively treat any disease.

The pharmaceutical compositions of described herein can be administered in any conventional manner by any route where they are active. Administration can be systemic or local. For example, administration can be, but is not limited to, parenteral, subcutaneous, intravenous, intramuscular, intraperitoneal, transdermal, oral, buccal, ocular, intravaginally, or inhalation. In certain embodiments, the administration may be systemic by intravenous injection, and in other embodiments, the administration to subjects exhibiting cancer may be by intratumoral injection. In some embodiments, the pharmaceutical composition may be prepared in the presence or absence of stabilizing additives that favors extended systemic uptake, tissue half-life, and intracellular delivery. Thus, modes of administration for the compounds of the present invention either alone or in combination with other pharmaceuticals can be injectable, including short-acting, depot, implant, and pellet forms injected subcutaneously or intramuscularly. In some embodiments, an injectable formulation including SWCNT complexes may be deposited to a site of the diseased tissue, such as, for example, in some embodiments, the pharmaceutical composition may be administered directly to tumorigenic tissue. In other embodiments, the pharmaceutical composition may be administered systemically by, for example, intravenous injection.

The frequency of administration may vary depending on the disease indication being treated and the patient's response to the treatment. For example, in some embodiments, a pharmaceutical composition including SWCNT complexes may be administered at least once ever 12 hours, at least once every 24 hours, at least once every 48 hours, or at least once every 72 hours. Without wishing to be bound by theory, the half-life of the SWCNT complexes may be relatively short in circulation; however, despite this limited half-life in circulation, the SWCNT complexes may retain activity in affected tissue for at least about 24 hours following administration. For example, in some embodiments, a SWCNT/complex may be detectable in the blood stream of a patient to whom a pharmaceutical composition including a SWCNT/complex for less than about 30 minutes or less than about 15 minutes, but reduction in expression of a target gene may be observed for up to at least 12 hours or at least 24 hours. Thus, while the half-life of the SWCNT/complex may be relatively short, sufficient levels of the gene or gene product payload can be delivered to affected tissue to adequately from a single therapeutically effect dose once every 12 hours or 24 hours. The frequency of administration of a pharmaceutical composition including a SWCNT/siRNA complex may, therefore, be reduced based on the effect rather than concentration of the SWCNT/complex in circulation.

The amount of SWCNT complexes in circulation following administration may be further effected by introduction of a surfactant into the pharmaceutical composition. For example, in some embodiments, the half-life of a pharmaceutical composition in circulation may be increased by providing SWCNT complexes having up to about a 10:1 molar ratio of a surfactant to the bioactive agent, and in other embodiments, the ratio of surfactant to bioactive agent in SWCNT complexes having extended half life may be from about 8:1 to about 1:1, or about 7:1 by molar ratio. Without wishing to be bound by theory, SWCNT complex having a high ratio of surfactant to bioactive agent may exhibit a half-life in circulation of about 30 minutes or more, about 15 minutes or more, about 10 minutes or more, or about 5 minutes or more. In still other embodiments, the surfactant agent may be provided at less than a 1:1 ratio as compared to the bioactive agent. For example, in some embodiments, the ratio of surfactant agent to bioactive agent may be about 1:10, and in other embodiments, about 1:2 to about 1:8, or about 1:7. In embodiments with a low ratio of surfactant to bioactive agent, almost no SWCNT complexes may be detectable in circulation following administration; however, the reduced half-life for the SWCNT complexes in circulation may not effect of the pharmaceutical composition on the target tissue.

Yet further embodiments are directed to methods for preparing a SWCNT complexes including the steps of combining a bioactive agent in an aqueous solution with SWCNT and sonicating the SWCNT/bioactive agent solution. In some embodiments, the bioactive agent may be mRNA, and the concentration of mRNA in the aqueous solution, in such embodiments, may be up to about 100 μM, or in some embodiments, from about 5 μM to about 50 μM or about 10 μM to about 30 μM. In particular embodiments, the concentration of bioactive may be about 20 μM. In some embodiments, the SWCNT that are combined with the bioactive agent in the aqueous solution may be provided in an aqueous solution at a concentration of about 1 ppm to about 10 ppm or up to concentration of about 500 mg/mL, and when combined with the bioactive the concentration of SWCNT in the SWCNT/bioactive agent solution may be about 10 μg/mL to about 500 μg/mL or about 25 μg/mL to about 300 μg/mL. In some embodiments, the SWCNT/bioactive agent solution may further include one or more surfactants. For example, in certain embodiments, a surfactant may be added to the aqueous solution to provide a final concentration (w/v) of surfactant of about 1% to about 12%, about 2% to about 9%, or about 3% of the total solution.

The aqueous solution of various embodiments may be any buffer known in the art that is useful during sonication and may include any number of chemical additives. For example, in some embodiments, the aqueous solution may be a buffer solution of about 100 mM KCl, 30 mM HEPES-KOH, and 1 mM MgCl₂ or 0.9% NaCl. The pH of such buffer solutions may be approximately neutral, for example, from about 6.5 to about 8.0. In particular embodiments, the aqueous solution may be suitable for in vivo administration of the SWCNT complexes. As such, in some embodiments, salt and pH concentrations may be within physiological ranges, and in other embodiments, the aqueous solution may be sterilized by known methods. The SWNCT complexes in the aqueous solution may, therefore, be administered immediately following sonication.

Sonication may be carried out using any sonication device known in the art, and the parameters for sonication may vary depending, for example, on the type of bioactive agent being associated with the SWCNT. For example, in embodiments in which an oligonucleotide encoding a therapeutic protein is associated with SWCNT, the SWCNT/oligonucleotide solution is sonicated under low shear conditions in a plurality of bursts for a period of time sufficient to solubilize and prevent aggregation of the SWCNT while resulting in low shear as defined as undetectable or insignificant shear by length measurement. Shear is readily determined by gel electrophoresis with confirmation of the functional viability of a large polynucleotide of a given size by assaying the in vitro transfection efficiency of a marker gene of similar size. In some embodiments of complexation with a large polynucleotide, sonication was performed in 60 second bursts with no intervals between bursts for a total of 30 minutes. In some embodiments, the method for sonication may include two 30 minute bursts for a total of 60 minutes of sonication. In particular embodiments, the temperature of the samples during sonication may be maintained at about 10° C. during the sonication, and the samples may be placed on ice following sonication. The sonicator settings during sonication may vary but are determined to achieve solubilization without shearing the large polynucleotides.

For example, in some embodiments, the sonicator effectively utilized was a high intensity, focused, computer controlled acoustic shock sonicator. In one embodiment a Covaris S2 sonicator employing acoustic waves was used and was set with acoustic low shear conditions by setting the Treatment Parameters of a Duty Cycle of 20%, an Intensity of 1 (out of a range from 0.1-10) with 200 Cycles/Burst (out of a range from 50-1000) for 30 cycles. Using an acoustic wave sonicator the acoustic power used could be up to 80 watts, could be in the range of 1 to 60 watts or could be in the range of 8-24 watts.

The methods of some embodiments may further include the step of removing insoluble materials from the aqueous solution following sonication. The step of removing insoluble materials may be carried out by any means known in the art. For example, in some embodiments, insoluble materials may be removed by filtration, and in other embodiments, insoluble materials may be removed by centrifugation using parameters determined empirically to be sufficient, such as for example, 16,000×g for 10 minutes.

Various modifications of the invention, in addition to those described herein, will be apparent to those skilled in the art from the foregoing description. Such modifications are intended to fall within the scope of the appended claims.

### EXAMPLES

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples. The following examples are for illustrative purposes only and are not to be construed as limiting the invention in any manner.

### Example 1: SWCNT Transfection System for Primary Cells

The largest component of PBMC are T cells and the present data show that SWCNT can transfect PBMC both in vitro and in vivo, and that SWCNT can complex with plasmid DNA, and are able to transport their payloads into the nucleus. The utility of SWCNT as a platform for transfection of genetic material into primary human T cells is further demonstrated. As a model system, improved gene delivery platforms ultimately for the preparation of CAR T cells for the treatment of B cell leukemia and other appropriate cancers was utilized.

The present inventors have overcome the prior reported drawbacks associated with such it-stacking (attractive, noncovalent interactions between aromatic rings) complexes and has clearly identified how these complexes can be prepared along with the stability of these complexes. Another drawback previously associated to the it-stacking complexes is that once dissociated from the complex, the carbon nanotube (CNT) by itself is not soluble in aqueous systems and tends to form hydrophobic aggregates that precipitate. The present inventors have determined that this precipitation does not occur in vivo since the dissociation of the pDNA or RNA is desired and usually occurs through an exchange process with other biological molecules that maintain the solubility of the SWCNT. It was previously reported that such drawbacks severely impaired the ability of these non-covalent π-stacking complexes to be used for the delivery of the molecules complexed to the carbon nanotubes in biological systems. Certain of the present inventors previously determined that siRNA can be effectively delivered in vivo. As provided herein, formulations are disclosed that enable formation of π-stacking complexes with large polynucleotide that can penetrate into primary T cells in vitro or following intravenous injection. Thus, provided herein are formulations of complexed SWCNT that can readily transfect hard-to-transfect PBMC in vitro and in vivo versus the prior demonstrations of immortalized cells in vitro or other tissues that are not hard to transfect in vivo. Provided herein are SWCNT formulations able to transfect human primary T cells with plasmid DNA and RNA providing method for delivery of genes or gene products that eliminates the need to use a viral delivery platform.

Preparation of Noncovalent Complexes of SWCNT with T Cell Receptor mRNA.

Materials: 1.14 mg pristine SWCNT (HiPco, Rice University, Lot# R0559); 0.571 mg TCR RNA (TCR alpha RNA 2.4 mg/mL stock solution), (TCR beta RNA 2.0 mg/mL stock solution)(U PENN); 5 k L-PEG, in this case, 14:0 PEG5000 PE (1,2-dimyristoyl-sn-glycero-3-phosphoethanolamine-N-[methoxy (polyethylene glycol)-5000] (ammonium salt, cat. #88010 Avanti Polar Lipids) in a stock solution at 20 mg/ml of DMSO); 20 mg DEPT-Treated water (Ambion); NaCl, 0.9% aqueous solution (Horpira); Dimethyl Sulfoxide (Fisher); Sonicator (Covaris S2); 12×24 mm round bottom glass tube with cap, sterilized; 1.7 mL microcentrifuge tubes (Eppendorf, 12-15 sterile); 0.5 mL centrifugal filters (Amicon Ultra 100K, Millipore); NanoDrop 1000 Spectrophotometer (Thermo Fisher Scientific); NS2 NanoSpectralyzer (Applied NanoFluorescence); 1.5 mL disposable plastic cuvettes.

Procedures: All solutions were kept sterile by working in a sterile tissue culture hood and solutions were kept on ice between all steps. Prior to beginning complex preparation, the Covaris S2 Sonicator was adjusted to 10° C. and allowed to acclimatize. Centrifuge temperature was set to 4° C.

Pristine SWCNT (1.14 mg) were placed into 12×24 mm sterilized round bottom glass tubes. A stock solution of the 5 k L-PEG in dimethyl sulfoxide (DMSO) was prepared by dissolving 20 mg of 5 k L-PEG in 1 mL DMSO. A solution of 0.579 mg/mL RNA was prepared diluting appropriate amount of RNA stock solution with 0.9% NaCl and measuring the RNA concentration using NanoDrop. The RNA solution (0.986 mL) was added to glass tube containing the SWCNT. The RNA/SWCNT solution was sonicated using a Covaris Sonicator set at 10% power at 10° C. for 30 min (60 second pulses without pauses, 30 cycles). Following the 30 minute sonication, the 5 k L-PEG stock solution (0.014 mL) was added to the SWCNT/RNA solution to produce a final 5 k L-PEG concentration of 50 μM and the mixture sonicated for another 30 min (60 second pulses without pauses, 30 cycles) for a total sonication time of 60 min. The final RNA concentration after addition of the 5 k L-PEG was 0.571 mg/mL, and the final volume was 1 mL.

The SWCNT/RNA/PEG mixture was transferred into a sterile Eppendorf tube and centrifuged at 16,000×g for 10 min at 4° C. The resulting supernatant was transferred into a second sterile Eppendorf tube and centrifuged for another 10 min with the same settings. The resulting supernatant containing the suspension of SWCNT/RNA/PEG complex was transferred into a third sterile Eppendorf tube.

To measure the final SWCNT concentration, a 25 μL sample of the complex solution was diluted with 0.9% NaCl to a final volume of 400 placed in a cuvette and the NIR and absorbance spectra were measured using the NanoSpectralyzer NS2. The resulting SWCNT concentration was in a range of 0.03-0.09 mg/mL. The SWCNT/RNA/PEG complex solution was stored at 4° C. until use. The solution was stable for at least 1 month.

To prepare a 3.5 mL of SWCNT/RNA/PEG solution, initial amounts of SWCNT were proportionally increased to 2 mg, RNA to 4 mg, PEG to 0.994 mg, and total volume to 3.5 mL. For sonication, 5 mL Covaris tubes were used for the same sonication time although the sonication power was increased to 30%.

Near infrared (NIR) fluorescence spectroscopy indicated that the sample contained predominantly individually suspended SWCNT rather than nanotube aggregates.

The near infrared (NIR) emission spectrum of the mRNA-suspended SWCNT was measured using 658 nm excitation in a model NS1 NanoSpectralyzer (Applied NanoFluorescence, Houston, Tex.). NIR fluorescence microscopy was performed using a custom-built apparatus containing diode laser excitation sources emitting at 658 and 785 nm. Individual SWCNT internalized into cells were imaged with a custom-built NIR fluorescence microscope using 785 nm excitation, a 60× oil-immersion objective, and a 946 nm long-pass filter in the collection path. Bright field images were taken using the 60× objective.

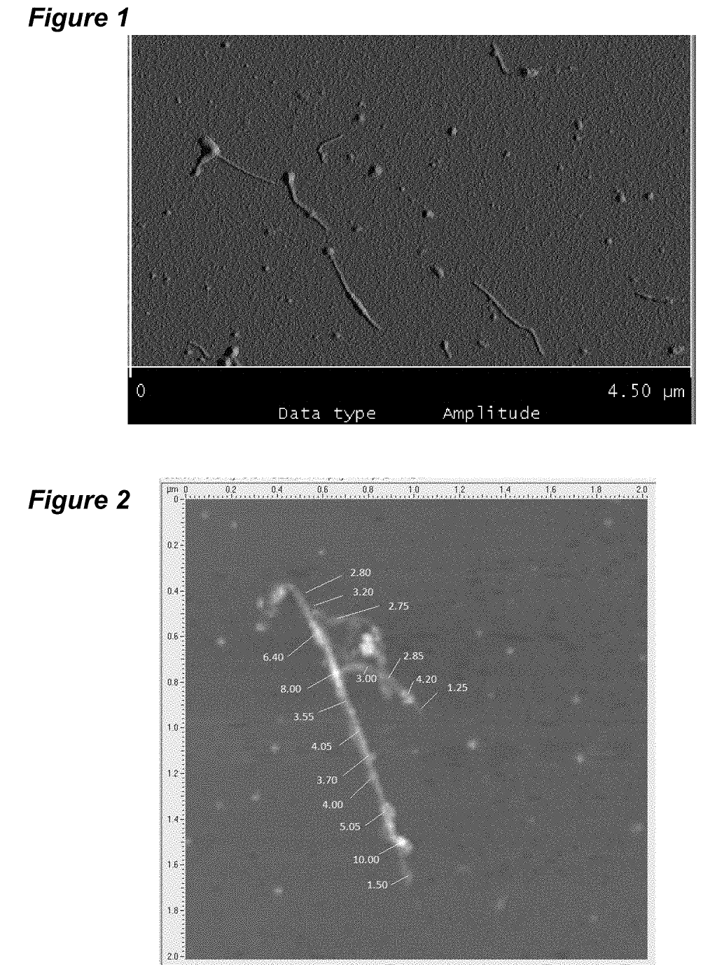

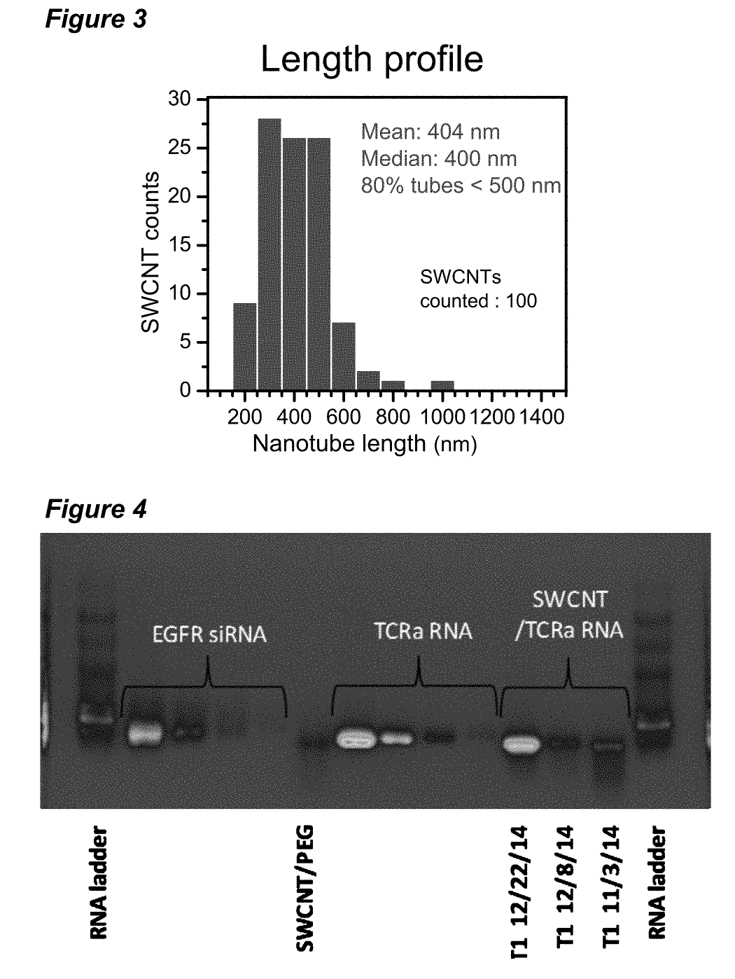

The pristine SWCNT are made water-compatible or stable in solution by coating with the mRNA. The computer controlled focused-ultrasonicator, such as the Covaris S2, provided high frequency, accurately focused, and specifically targeted acoustic energy, into the sample vesicle. These characteristics enable a highly reproducible and isothermal process, reducing experimental variation and heat induced damage to the sample. By processing in a closed vessel, the risk of contamination between samples was alleviated. Additionally the power was closely controllable to prevent shearing of the oligonucleotide samples during the “solubilization” of the pristine SWCNT by the polynucleotide. Attempts to first coat the SWCNT with surfactants such as PEG-lipid macromolecules prior to addition of the polynucleotide required longer sonication times and resulted in SWCNT that were significantly reduced in length as compared with SWCNT that were first coated or “solubilized” with oligonucleotides. SWCNT/oligonucleotide complexes prepared by first coating the SWCNT with the oligonucleotide followed by addition of the surfactant resulted in a more uniform length distribution and higher oligonucleotide coating. These suspensions displayed strong NIR fluorescence between approximately 900 and 1600 nm. FIG. 1 depicts an Amplitude profile by Atomic Force Microscopy image of a SWCNT/TCRα RNA/PEG complex. FIG. 2 depicts the height profile of the same complex and FIG. 3 depicts the length analysis. The images show the SWCNT well coated with mRNA.

Agarose gel electrophoresis was used to determine if the RNA was sheared through processing or if it remained intact. Samples defined as T1 11/3/14 and 12/8/14 were prepared using previously reported siRNA sonication procedures using 100% power and 15 second pulses for a total of 4 min. Poor SWCNT loading was observed and the RNA was sheared as shown in FIG. 4. Altering the sonication procedure to use 10% power but longer sonication time for overall higher energy input provided a SWCNT/RNA complex with superior RNA loading and the RNA remained intact (T1 12/22/14)

### Example 2: Human Primary T Cell Culture and Cellular Transfection with SWCNT/MRNA Complexes

Human T-cells were thawed and suspended in RPMI-1640 complete media at 106/mL and placed in a 96 well plate with confirmation of viability. If not stimulating, PBMC were transfected after ˜1-2 hours after thaw. If stimulating in vitro following the methodology of eBioscience:T-cell activation, transfection was performed at 72 hours after the stimulation protocol when PBMC would have responded to stimulation (usually verified by Cell Titer Blue signal) and left with the SWCNT for either 6 or 24 hours.

SWCNT/mRNA complex formulated with 50 μM or 1600 μM 5 k L-PEG or vehicle were added into wells with a total 5 to 40 μg of SWCNT. Cells were incubated for 6 or 24 hr. The cells were then removed and washed with PBS to remove any excess SWCNT and replated in a 96 well plate.

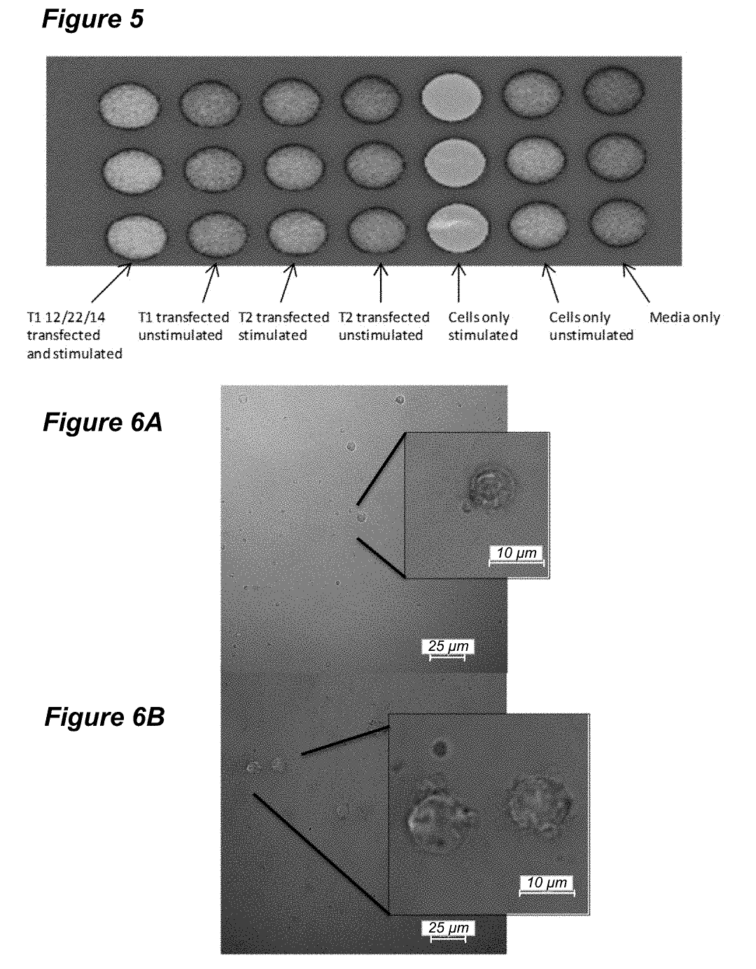

The SWCNT/RNA/PEG complex T1 12/22/14 (prepared with 50 μM PEG), was used to transfect primary PBMC and was compared to a second SWCNT/RNA/PEG complex prepared with 1600 μM PEG, T2. Cells were imaged using Cell Titre Blue to measure cell growth as shown in FIG. 5. The results of the study showed that T1 12/22/14 provided better transfection in vitro into the PBMC than the T2 solution. Additionally T1 12/22/14 transfected T-cells could be stimulated to grow as shown in FIG. 5. Cells were isolated and then imaged using near infrared microscopy to identify SWCNT in the T-cells.

The advantages of using SWCNT to deliver large polynucleotides include resistance to nuclease digestion, the stability of the complex in the presence of serum, efficient transfection and evasion of endosomal capture, reducing the amount of oligonucleotide required for systemic delivery to produce tumor target KD, the lack of toxicity of the complex and rapid elimination from the body of the de-cargoed nanotube carrier. SWCNT/polynucleotide complexes readily penetrate cell membranes by a process described as nanospearing, i.e. tiny needles passing through the cell membrane, and by endocytosis. Importantly, the SWCNT exit the cell by the same processes and excretion occurs through the kidney and in the bile.

SWCNT Transfection of PBMC with Cy-3-siRNA:

The ability of SWCNT complexed with Cy-3 labeled siRNA to transfect human PBMC was investigated in vitro. Three formulations of SWCNT/siRNA targeting EGFR were prepared and added to PBMC in culture and confocal images were obtained at 2, 6 and 24 hr following transfection. FIG. 6A provides confocal images of Human PBMC exposed to Cy-3 siEGFR for 24 hr. FIG. 6B provides confocal images of human PBMC exposed to SWCNT complexed siEGFR formulated for in vitro transfection for 24 hr. The insert illustrates single cells at 10× magnification.

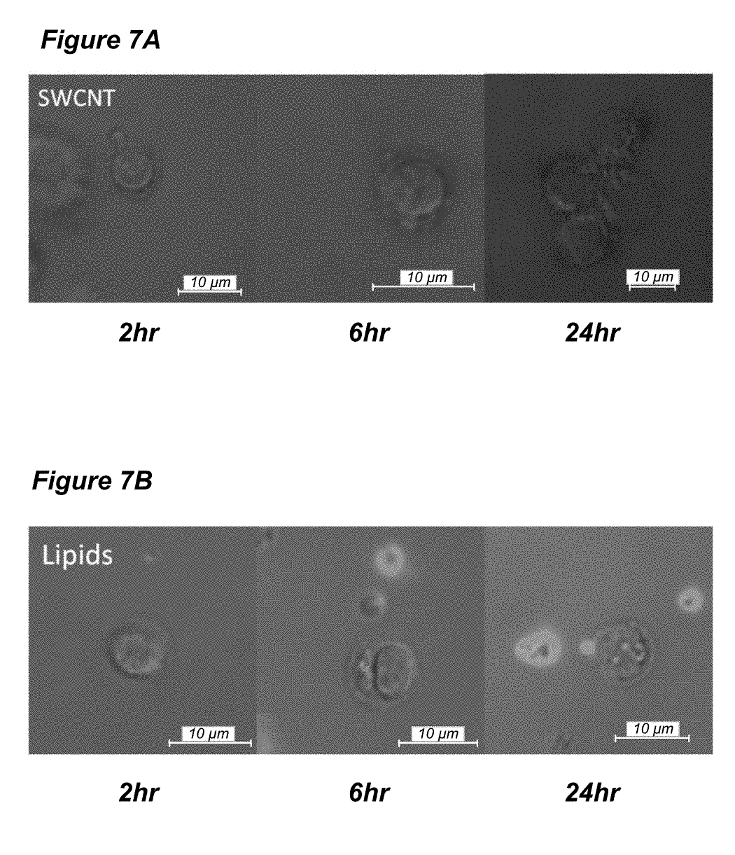

FIGS. 6A and B show the superior SWCNT transfection (B) over the uncomplexed Cy-3 siRNA solution (A) with the label distributed throughout the cytoplasm as well as into the nucleus. There was no Cy3 signal in cells at any of the time points when not complexed to SWCNT. FIGS. 7A and B show the comparison between SWCNT and a commercial liposome preparation of the same concentration as delivered by SWCNT. FIG. 7A provides confocal images of human PBMC exposed to SWCNT/Cy-3 siEGFR formulated for in vitro delivery (5 μg/mL SWCNT) showing intracellular delivery. FIG. 7B provides confocal images of human PBMC exposed to Cy-3 siEGFR in a liposomal formulation for 2, 6 and 24 hr. Color images showed that without the SWCNT, agglomerated liposomes were located extracellularly. Cells exposed to SWCNT/Cy-3 siRNA showed cellular uptake at 2 hr and this increased over a 24 hr period. Lipid transfection was unsuccessful even after a 24 hr exposure and color images of FIG. 6B showed that particles agglomerated outside the cells over this time period.

SWCNT Transfection of PBMC with siRNA In Vivo:

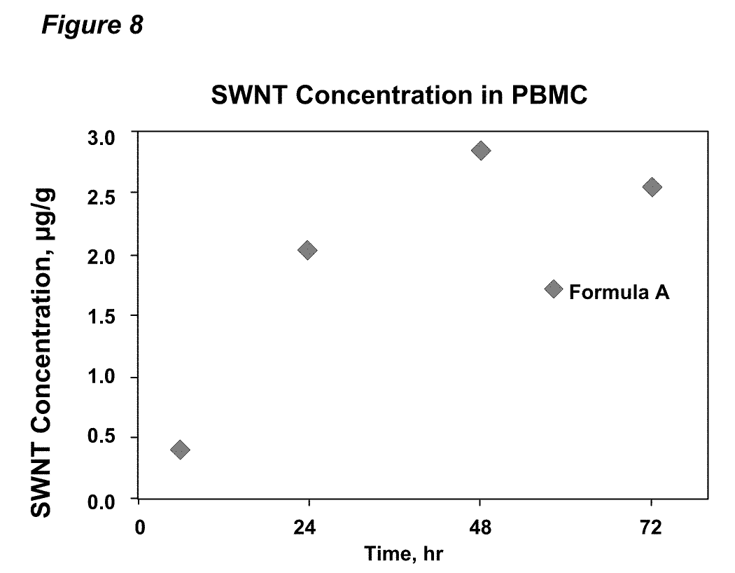

Pharmacokinetic and biodistribution analyses were conducted in C57BL6 mice to evaluate how different formulations of SWCNT/siRNA affect t½ and tissue distribution. In these studies, whole blood was collected and separated PBMCs to determine SWCNT distribution. Following a 100 μg SWCNT dose administered intravenously, blood was taken at 6, 24, 48 and 72 hr and used to determine SWCNT levels in plasma and PBMC. These studies showed that the SWCNT/siRNA formulations together with the surfactant 14:0 PEG5000 PE (1,2-dimyristoyl-sn-glycero-3-phosphoethanolamine-N-[methoxy (polyethylene glycol)-5000 incorporated at 9% were cleared from the plasma with a peak 0.004 mg/mL concentration by 6 hr and were undetectable at 24 hr and times later. Conversely in PBMC the SWCNT concentrations were 0.419, 2.05, 2.85, 2.56 μg/g at 6, 24, 48, 72 hr respectively giving approximately 441, 2149, 2994, 2690 SWCNT per each PBMC at 6, 24, 48, 72 hr, respectively as shown in FIG. 8. The “formula A” of the Figure is a 1600 uM solution of L-PEG complexed to the SWCNT/siRNA somplex and then washed to remove excess siRNA and PEG prior to i.v. administration.”

SWCNT Transfection of PBMC with Large Polynucleotides In Vitro and In Vivo:

Large polynucleotides are subject to shear and formulations able to protect the polynucleotide from shear are critical to delivery of these molecules. Several formulations and methods of formation of complexes of large polynucleotides with pristine SWCNT were tested. These included three mRNA preparations and two DNA preparations, all of which were pristine—non-functionalized polynucleotides. One mRNA tested was a 996 bp polynucleotide encoding an Enhanced Green Fluorescent Protein (“EGFP”) mRNA (5-methylcytidine, pseudouridine). Two further mRNAs tested were approximately 1500 bp mRNAs containing polyA tails and encodingl G4 TCR (against NY-ESO-1) alpha and 1G4 TCR (against NY-ESO-1) beta. Each T cell Receptor (“TCR”) is a hetero-dimer composed of either α and β chains, or γ and δ chains. Here an alpha-beta chain heterodimer was encoded. The 1G4 TCR is a high affinity TCR that was generated by phage-display directed evolution to recognize the so-called NY-ESO-1157-165 peptide (SLLMWITQC) tumor-associated peptide antigen in the context of a human leukocyte antigen (pHLA*0201). See Li, Y. et al. “Directed evolution of human T-cell receptors with picomolar affinities by phage display” Nat Biotechnol. 2005 March; 23(3):349-54. The two large polynucleotide DNAs tested where a 5657 bp plasmid vector encoding Green Fluorescent Protein (“GFP”) and the 717 bp cDNA open reading frame (ORF) contained in the vector.

The large polynucleotides were complexed with pristine SWCNT with and without the 5 k L-PEG 5000 according to Example 1. Specifically, pristine SWCNT were complexed with the large polynucleotides at 10 μM, 50 μM and 1600 μM 5 k L-PEG prior to sonication. Sonication was performed at 100% power (Covaris S2 focused sonicator) of 15 second bursts with 45 second rest over 2 min, which is a setting that is effective for formation of viable siRNA complexes, compared with the following low power settings. For the low power settings, SWCNT were sonicated with the large polynucleotides (1 mL solution) with the Covaris S2 at 10% power, 10° C. for 30 min with no rest steps (30 cycles). Where 5 k L-PEG was included, further 5 k L-PEG was added and sonication was repeated for 30 minutes. For larger volume 3.5 mL solutions, the complexes were sonicated with the Covaris S2 at 30% power with other conditions held constant.

It was found that visually stable complexes were formed with or without the use of the L-PEG. However, in the absence of the L-PEG, no transfection of T-cells was obtained in vitro. Addition of the L-PEG was adjusted to obtain both a stable solution and high transfection efficiency. For example, it was found that the use of 10 μM 5 k L-PEG prior to sonication resulted in transfection of T-cells in vitro but the complexes were not as stable. Storage stability is an important parameter in development of a therapeutic formulation. Sonication sufficient to form stable solutions without SWCNT aggregation for siRNA/SWCNT complexes was found to result in shearing with large polynucleotides. Surprisingly, it was found that use of low power sonication at approximately 10-30% of full power but for approximately 10 times as long resulted in stable solutions with undetectable shear of the large polynucleotides. The data described above demonstrate the efficiency of SWCNT transfection in hard to transfect primary T-cells and support the use of this novel formulation in T-cell mediated gene therapy.

What is claimed is:

1. A method for delivering a large polynucleotide encoding a therapeutic protein to a patient in need thereof comprising:
isolating primary peripheral blood mononuclear cells (“PBMC”) from the patient;
generating a population of transfected autologous PBMC by transfecting the PBMC with a stable formulation comprising pristine large polynucleotides encoding therapeutic proteins that are non-covalently complexed to pristine single-walled carbon nanotubes under low shear conditions followed by formulating with a surfactant under low shear conditions; and
introducing the transfected autologous PBMC into the patient.

2. The method of claim 1, wherein introducing comprises intravenous injection.

3. The method of claim 1, wherein the patient is selected from a mammal, a mouse, and a human.

4. The method of claim 1, wherein the patient is a human cancer patient.

5. The method of claim 1, wherein the pristine large polynucleotides encode a Chimeric Antigen Receptor (CAR).

6. The method of claim 1, wherein the surfactant is selected from a polyalkylene oxide, PEG 5000, an L-PEG, 1,2-dimyristoyl-sn-glycero-3-phosphoethanolamine-N-[methoxy(polyethylene glycol)], poly(maleic anhydride-alt-1-octadecene)-poly(ethylene glycol) methyl ether, and combinations thereof.

7. The method of claim 6, wherein the L-PEG is a dimyristoyl modified PEG.

8. The method of claim 6, wherein the surfactant is 1,2-dimyristoyl-sn-glycero-3-phosphoethanolamine-N-[methoxy (polyethylene glycol)-5000].

9. The method of claim 1, wherein the stable formulation further comprising a pharmaceutically acceptable carrier selected from water, an isotonic salt solution, an isotonic sugar solution, polyethylene glycol (PEG), aqueous PEG solutions, liposomes, ethanol, organic solvents dissolved in isotonic aqueous solution, aqueous buffers, oils, and combinations thereof.

10. The method of claim 1, wherein the pristine single walled carbon nanotubes are prepared using a high pressure carbon-monoxide method (HiPco), in which high pressure carbon monoxide (CO) is disproportionated on iron (Fe) nanoparticles formed in a gas phase from iron pentacarbonyl (Fe(CO)₅) decomposition.

11. A method for delivering a large polynucleotide encoding a therapeutic protein to patient comprising:
preparing a stable formulation comprising polynucleotides non-covalently complexed to pristine single-walled carbon nanotubes with subsequent formulation with a surfactant under low shear conditions, wherein the stable formulation is adapted for uptake and expression by peripheral blood mononuclear cells (“PBMC”) after intravenous injection of the formulation into the patient.

12. The method of claim 11, wherein the surfactant is selected from a polyalkylene oxide, PEG 5000, an L-PEG, 1,2-dimyristoyl-sn-glycero-3-phosphoethanolamine-N-[methoxy(polyethylene glycol)], poly(maleic anhydride-alt-1-octadecene)-poly(ethylene glycol) methyl ether, and combinations thereof.

13. The method of claim 11, wherein the L-PEG is a dimyristoyl modified PEG.

14. The method of claim 11, wherein the surfactant is 1,2-dimyristoyl-sn-glycero-3-phosphoethanolamine-N-[methoxy (polyethylene glycol)-5000].

15. The method of claim 11, wherein the single walled carbon nanotubes are prepared using a high pressure carbon-monoxide method (HiPco), in which high pressure carbon monoxide (CO) is disproportionated on iron (Fe) nanoparticles formed in a gas phase from iron pentacarbonyl (Fe(CO)₅) decomposition.

16. A method for delivering an siRNA to patient comprising:
preparing a stable formulation comprising siRNA non-covalently complexed to pristine single-walled carbon nanotubes with subsequent formulation with a surfactant under low shear conditions, wherein the stable formulation is adapted for uptake by peripheral blood mononuclear cells (“PBMC”) of the patient.

17. The method of claim 16, wherein the surfactant is selected from a polyalkylene oxide, PEG 5000, 1,2-dimyristoyl-sn-glycero-3-phosphoethanolamine-N-[methoxy(polyethylene glycol)], poly(maleic anhydride-alt-1-octadecene)-poly(ethylene glycol) methyl ether, and combinations thereof.

18. The method of claim 17, wherein the surfactant is 1,2-dimyristoyl-sn-glycero-3-phosphoethanolamine-N-[methoxy (polyethylene glycol)-5000].

19. The method of claim 16, wherein the single walled carbon nanotubes are prepared using a high pressure carbon-monoxide method (HiPco), in which high pressure carbon monoxide (CO) is disproportionated on iron (Fe) nanoparticles formed in a gas phase from iron pentacarbonyl (Fe(CO)₅) decomposition.

20. The method of claim 16, wherein the stable formulation of pristine siRNA is first non-covalently complexed to pristine single-walled carbon nanotubes by sonication under low shear conditions and formulated with a surfactant at a final concentration (w/v) of about 1% to about 15% of surfactant.
